(19) United States
(12) Patent Application Publication
(10) Pub. No.: US 2023/0250094 A1
(43) Pub. Date: Aug. 10, 2023

(54) SMALL MOLECULE INHIBITORS OF TDP-43 ACTIVITY AND USES THEREOF

(71) Applicant: Arizona Board of Regents on Behalf of the University of Arizona, Tucson, AZ (US)

(72) Inventors: May Khanna, Tucson, AZ (US); Vijay Gokhale, Tucson, AZ (US); Liberty Francois-Moutal, Tucson, AZ (US); David Scott, Tucson, AZ (US); Haley Williams, Tucson, AZ (US); Judith Tello, Tucson, AZ (US); Niloufar Mollasalehi, Tucson, AZ (US)

(21) Appl. No.: 18/015,775

(22) PCT Filed: Jul. 15, 2021
(86) PCT No.: PCT/US2021/041852

§ 371 Date: Jan. 12, 2023

Related U.S. Application Data
(60) Provisional application No. 63/052,163, filed on Jul. 15, 2020.

(51) Int. Cl. C07D 413/12

(52) U.S. Cl. CPC C07D 413/12

(57) ABSTRACT

This invention is in the field of medicinal pharmacology. In particular, the present invention relates to pharmaceutical agents which function as inhibitors of TDP-43 activity. The invention further relates to methods of treating and/or ameliorating symptoms related to conditions associated with TDP-43 activity (e.g., neurodevelopmental disorders), comprising administering to a subject (e.g., a human patient) a composition comprising one or more pharmaceutical agents which function as inhibitors of TDP-43 activity.

### CROSS REFERENCE TO RELATED APPLICATION

This application claims priority to and the benefit of U.S. Provisional Application No. 63/052,163, filed Jul. 15, 2020, which is hereby incorporated by reference in its entirety.

### FIELD OF THE INVENTION

This invention is in the field of medicinal pharmacology. In particular, the present invention relates to pharmaceutical agents which function as inhibitors of TDP-43 activity. The invention further relates to methods of treating and/or ameliorating symptoms related to conditions associated with TDP-43 activity (e.g., neurodevelopmental disorders), comprising administering to a subject (e.g., a human patient) a composition comprising one or more pharmaceutical agents which function as inhibitors of TDP-43 activity.

### INTRODUCTION

Neurodegenerative diseases like Alzheimer's disease, Parkinson's disease, and amyotrophic lateral sclerosis are among the top 10 most fatal incurable diseases. As the global population ages, the incidence of these diseases will continue to increase. Current drugs targeting these diseases can slow progression and reduce the severity of the disease's symptoms, but they are not extremely effective at doing so, and none are able to cure the disease permanently.

Improved treatments for neurodegenerative diseases are desperately needed.

The present invention addresses this need.

### SUMMARY

Transactive response (TAR) DNA binding Protein-43 (TDP-43), a critical factor in neurodegenerative diseases including Amyotrophic Lateral Sclerosis (ALS) and Alzheimer's disease (AD), is involved in almost all aspects of RNA metabolism^{1,2}. TDP-43 consists of an N-terminal domain (NTD), two RNA recognition motifs (RRM1 and RRM2) and an unstructured glycine-rich domain.

The NTD is responsible for TDP-43 sub-cellular localization since it contains a nuclear localization signal (NLS)³⁻⁶ and one mitochondrial targeting sequence (M1)⁷. TDP-43 NTD has been shown to form dimers that can assemble into reversible higher-order oligomers, required for splicing activity⁸⁻¹¹ and contributing to liquid-liquid phase separation^{8,11}. Several studies have also hinted at the ability of TDP-43 NTD to both serve as a scaffold for nucleic acid binding and contribute to specificity towards certain nucleic acid sequences²⁻¹⁴, even though the nucleotide binding interface of the NTD remains to be determined.

Experiments conducted during the course of developing embodiments for the present invention targeted the NTD of TDP-43, involved in several neurodegenerative diseases. In silico docking of 50K compounds to the NTD domain of TDP-43 identified a small molecule

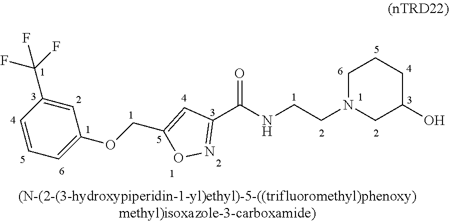 [SMILES: O=C(NCCN1CCCC(O)C1)c1cc(COc2cccc(C(F)(F)F)c2)on1]

(nTRD22) that bound to the N-terminal domain. Interestingly, nTRD22 caused allosteric modulation of the RNA binding domain (RRM) of TDP-43 resulting in a decreased ability to bind RNA in vitro. Moreover, incubation of primary motor neurons with nTRD22 induced a reduction of TDP-43 protein levels, similarly to TDP-43 binding deficient mutants and supporting a disruption of TDP-43 binding to RNA. Finally, nTRD22 was able to mitigate motor impairment in a Drosophila model of Amyotrophic Lateral Sclerosis. Such findings provide an exciting way of allosteric modulation of the RNA-binding region of TDP-43 through the N-terminal domain.

Accordingly, the present invention relates to pharmaceutical agents which function as inhibitors of TDP-43 activity. The invention further relates to methods of treating and/or ameliorating symptoms related to conditions associated with TDP-43 activity (e.g., neurodevelopmental disorders), comprising administering to a subject (e.g., a human patient) a composition comprising one or more pharmaceutical agents which function as inhibitors of TDP-43 activity.

In certain embodiments. the present invention provides compositions comprising one or more pharmaceutical agents capable of inhibiting TDP-43 activity. In some embodiments, the pharmaceutical agent capable of inhibiting TDP-43 activity is capable of one or more of the following: binding the N-terminal domain of TDP-43, engaging a pocket within the TDP-43 characterized by amino acids 548, A66, and N70, causing an allosteric modulation of the RNA binding domain (RRM) of TDP-43 thereby decreasing the ability of TDP-43 to bind RNA, and mitigating motor impairment in a subject suffering from or at risk of suffering from motor impairment.

In some embodiments, the present invention provides compositions comprising a pharmaceutical agent capable of binding the N-terminal domain of TDP-43. In some embodiments, the present invention provides compositions comprising a pharmaceutical agent capable of causing an allosteric modulation of the RNA binding domain (RRM) of TDP-43 thereby decreasing the ability of TDP-43 to bind RNA. In some embodiments, the present invention provides compositions comprising a pharmaceutical agent capable of decreasing the ability of TDP-43 to bind RNA. In some embodiments, the present invention provides compositions comprising a pharmaceutical agent capable of mitigating motor impairment in a subject suffering from or at risk of suffering from motor impairment. In some embodiments, the present invention provides compositions comprising a pharmaceutical agent capable of engaging a pocket within the TDP-43 characterized by amino acids S48, A66, and N70.

In some embodiments, the pharmaceutical agent capable of capable of inhibiting TDP-43 activity is

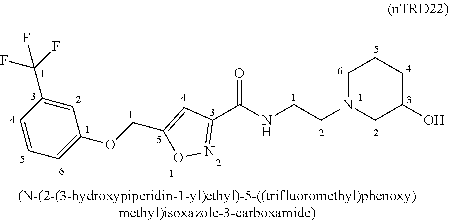 [SMILES: O=C(NCCN1CCCC(O)C1)c1cc(COc2cccc(C(F)(F)F)c2)on1]

or a structurally similar compound.

In some embodiments, the pharmaceutical agent capable of inhibiting TDP-43 activity is

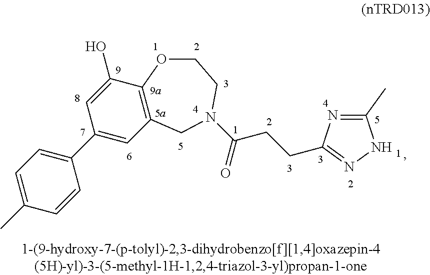

or a structurally similar compound.

In some embodiments, the pharmaceutical agent capable of inhibiting TDP-43 activity is

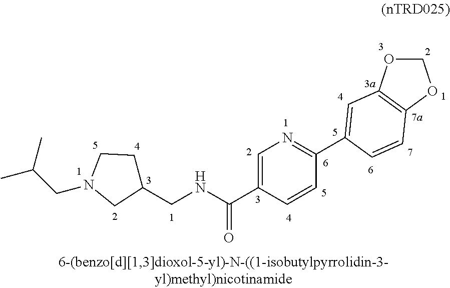 [SMILES: CC(C)CN1CCC(CNC(=O)c2ccc(-c3ccc4c(c3)OCO4)nc2)C1]

or a structurally similar compound.

In some embodiments, the pharmaceutical agent capable of inhibiting TDP-43 activity is

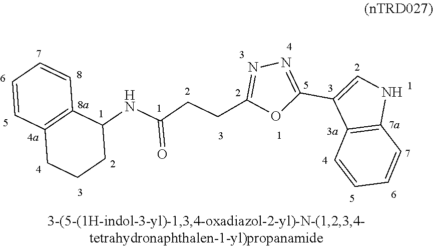 [SMILES: O=C(CCc1nnc(-c2c[nH]c3ccccc23)o1)NC1CCCc2ccccc21]

or a structurally similar compound.

In certain embodiments, the present invention provides methods for inhibiting TDP-43 activity in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for causing an allosteric modulation of the RNA binding domain (RRM) of TDP-43 in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for decreasing the ability of TDP-43 to bind RNA in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for treating, ameliorating and/or preventing a condition characterized with TDP-43 activity in a subject, comprising administering to the subject a composition comprising a pharmaceutical agent capable of inhibiting TDP-43 activity (e.g., nTRD22, nTRD013, nTRD025, nTRD027). In some embodiments, the subject is a human subject. In some embodiments, the subject is suffering from or at risk of suffering from a neurodevelopmental disorder. In some embodiments, the subject is a human subject suffering from or at risk of suffering from one or more of amyotrophic lateral sclerosis (ALS) or Alzheimer's disease (AD). In some embodiments, the method further comprises administration of a pharmaceutical agent known to be effective in treating a neurodevelopmental disorder (e.g., ALS, AD).

In certain embodiments, the present invention provides methods for treating, ameliorating and/or preventing one or more symptoms related to a condition characterized with TDP-43 activity in a subject, comprising administering to the subject a composition comprising a pharmaceutical agent capable of inhibiting TDP-43 activity (e.g., nTRD22, nTRD013, nTRD025, nTRD027). In some embodiments, the subject is a human subject. In some embodiments, the subject is suffering from or at risk of suffering from a neurodevelopmental disorder. In some embodiments, the subject is a human subject suffering from or at risk of suffering from one or more of amyotrophic lateral sclerosis (ALS) or Alzheimer's disease (AD). In some embodiments, the one or more symptoms include motor impairment. In some embodiments, the method further comprises administration of a pharmaceutical agent known to be effective in treating a neurodevelopmental disorder (e.g., ALS, AD).

In certain embodiments, the present invention provides kits comprising (1) a composition comprising a pharmaceutical agent capable of inhibiting TDP-43 activity (e.g., nTRD22, nTRD013, nTRD025, nTRD027), (2) a container, pack, or dispenser, and (3) instructions for administration.

### BRIEF DESCRIPTION OF THE DRAWINGS

FIG. 1. Docking of small molecules on N-terminal domain of TDP-43. Top 10 compounds (green, sticks and balls representation) from in-silico docking on TDP-43-NTD (A) NMR structure (2n4p¹²) and (B) crystal structure (5mdi⁸).

FIG. 2. STD-NMR screening for compounds. A. Two different constructs were used for STD-NMR screening. 1D ¹H STD NMR showing on-resonance difference spectrum of 500 μM of compounds nTRD09-16 (B) or nTRD19-28 (C) with 5 μM TDP43₁₋₂₆₀. D. Positive hits from the first round of STD-NMR screening were tested against TDP43₁₀₂₋₂₆₉ in the same conditions. Asterisks indicate positive signal.

FIG. 3. Modeling of binding and affinity of nTRD22 for TDP-43. A. Structure of N-(2-(3-hydroxypiperidin-1-yl)ethyl)-5-((3-(trifluoromethyl)phenoxy)methyl)isoxazole-3-carboxamide (nTRD22). B. 2D representation of nTRD22 binding pocket in TDP-43 NTD. C. MST values from thermographs of NT-647 labeled TDP43₁₋₂₆₀ in presence of increasing concentrations (0.03 μM-1 mM) of nTRD22 were used to determine dissociation constant for binding of nTRD22 to TDP43₁₋₂₆₀. Apparent Kd of 145±3 μM. Data is presented as mean±SD (n=3).

FIG. 4. A. Superposition of 1H-15N heteronuclear single quantum correlation spectroscopy (HSQC) spectra of ¹⁵N-labeled human TDP43₁₋₂₆₀ (100 μM), free (blue) and in complex with nTRD22 with different ratios. Close-up of shifts around TDP43 residues from TDP43₁₋₂₆₀ C173 (B) and G148 and G110 (C) or from TDP43₁₀₂₋₂₆₉ C173 D) and G148 and G110 (E). F. Chemical shift changes for assigned residues of the ¹⁵N-labeled TDP-43₁₋₂₆₀ (RRM portion only) upon complex formation with nTRD22. The average chemical shift changes of cross-peaks were calculated as described in the Methods section. The horizontal line (0.45 ppm) is the threshold, calculated as previously described¹⁷, above which a shift is considered significant with 1 sigma and the dotted line is set to 2 sigma G. The chemical shift difference observed on TDP43₁₋₂₆₀ were mapped on TDP43₁₀₂₋₂₆₉ (PDB ID: 4bs2¹⁵).

FIG. 5. RNA/TDP-43 disruption by nTRD22. A concentration-dependent curve was obtained for nTRD22's disruption of nucleic acid-TDP-43₁₋₂₆₀ interaction at a single RNA concentration (0.6 nM). Data is represented as mean±SEM (n=3).

FIG. 6. nTRD22 effect on primary cortical neurons. Confocal images of rodent primary cortical neurons with transfected TDP43-EGFP with A. DMSO, or B. 100 M nTRD22. Quantitation of GFP signal in TDP-43-EGFP (C) or EGFP (D) transfected primary neurons treated with increasing concentration of nTRD22 or DMSO. Data is presented as mean±SEM (n≥183 neurons from 8 technical and 3 biological replicates). Statistical difference was assessed by a Kruskal-Wallis test (*** p<0.001; **p=0.01).

FIG. 7. nTRD22 mitigates motor defects in a Drosophila model of ALS overexpressing TDP-43. A. Principle of the sensitized version of the negative geotaxis assay²⁰. Flies are transferred without anesthesia to a wax-sealed glass graduated cylinder. Flies are tapped to the bottom, and their subsequent climbing activity is quantified for 2 min. The number of flies crossing the target line (red) at each time point chosen (every 10 sec) is recorded. B. Fly food supplemented with nTRD22 (50 μM) resulted in increased motor performance compared to naïve or DMSO (0.05%) treated flies. (n=60, **** p<0.0001, Two-Way ANOVA).

FIG. 8: Sitemap of TDP-43 NTD structures. Surface representation of docking pockets (blue) found using SiteMap on the (A) average NMR structure (PDB ID: 2n4p¹⁰) or (B) crystal structure (PDB ID: 5mdi⁴).

FIG. 9: Superposition of ¹H-¹⁵N heteronuclear single quantum correlation spectroscopy (HSQC) spectra of ¹⁵N-labeled human TDP43₁₋₂₆₀ (100 μM), with DMSO (blue) and in complex with positive nTRD binders (400 μM) in red.

FIG. 10: Average normalized chemical shift changes for assigned residues of the ¹⁵N-labeled TDP-43₁₋₂₆₀ (RRM portion only) upon complex formation with nTRD22. The average chemical shift changes of cross-peaks were calculated as described in the Methods section. Data: Mean±SD (n=3).

FIG. 11: Fly food supplemented with nTRD22 (50 μM) resulted in increased motor performance compared to DMSO (0.05%) treated flies in young individuals (5-6 days post eclosion; dpe) as well as aged individuals (19-20 dpe). (n=60, **** p<0.0001, Two-Way ANOVA).

FIG. 12: Superposition of ¹H-¹⁵N heteronuclear single quantum correlation spectroscopy (HSQC) spectra of ¹⁵N-labeled human TDP43₁₀₂₋₂₆₉ (100 μM), free (black) and in complex with TDP43 NTD with different ratios.

### DETAILED DESCRIPTION OF THE INVENTION

Experiments conducted during the course of developing embodiments for the present invention targeted the NTD of TDP-43, involved in several neurodegenerative diseases. In silico docking of 50K compounds to the NTD domain of TDP-43 identified a small molecule

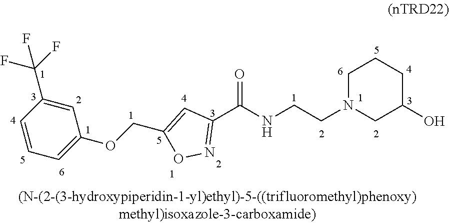 [SMILES: O=C(NCCN1CCCC(O)C1)c1cc(COc2cccc(C(F)(F)F)c2)on1]

that bound to the N-terminal domain. Interestingly, nTRD22 caused allosteric modulation of the RNA binding domain (RRM) of TDP-43 resulting in a decreased ability to bind RNA in vitro. Moreover, incubation of primary motor neurons with nTRD22 induced a reduction of TDP-43 protein levels, similarly to TDP-43 binding deficient mutants and supporting a disruption of TDP-43 binding to RNA. Finally, nTRD22 was able to mitigate motor impairment in a Drosophila model of Amyotrophic Lateral Sclerosis. Such findings provide an exciting way of allosteric modulation of the RNA-binding region of TDP-43 through the N-terminal domain.

Accordingly, the present invention relates to pharmaceutical agents which function as inhibitors of TDP-43 activity. The invention further relates to methods of treating and/or ameliorating symptoms related to conditions associated with TDP-43 activity (e.g., neurodevelopmental disorders), comprising administering to a subject (e.g., a human patient) a composition comprising one or more pharmaceutical agents which function as inhibitors of TDP-43 activity.

In certain embodiments. the present invention provides compositions comprising one or more pharmaceutical agents capable of inhibiting TDP-43 activity. In some embodiments, the pharmaceutical agent capable of inhibiting TDP-43 activity is capable of one or more of the following: binding the N-terminal domain of TDP-43, engaging a pocket within the TDP-43 characterized by amino acids S48, A66, and N70, causing an allosteric modulation of the RNA binding domain (RRM) of TDP-43 thereby decreasing the ability of TDP-43 to bind RNA, and mitigating motor impairment in a subject suffering from or at risk of suffering from motor impairment.

In some embodiments, the present invention provides compositions comprising a pharmaceutical agent capable of binding the N-terminal domain of TDP-43. In some embodiments, the present invention provides compositions comprising a pharmaceutical agent capable of causing an allosteric modulation of the RNA binding domain (RRM) of TDP-43 thereby decreasing the ability of TDP-43 to bind RNA. In some embodiments, the present invention provides compositions comprising a pharmaceutical agent capable of decreasing the ability of TDP-43 to bind RNA. In some embodiments, the present invention provides compositions comprising a pharmaceutical agent capable of mitigating motor impairment in a subject suffering from or at risk of suffering from motor impairment. In some embodiments, the present invention provides compositions comprising a pharmaceutical agent capable of engaging a pocket within the TDP-43 characterized by amino acids S48, A66, and N70.

In some embodiments, the pharmaceutical agent capable of capable of inhibiting TDP-43 activity is

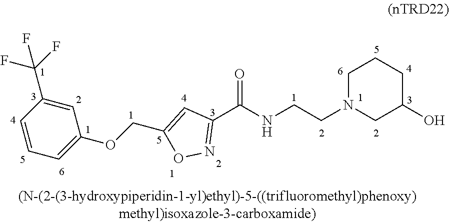 [SMILES: O=C(NCCN1CCCC(O)C1)c1cc(COc2cccc(C(F)(F)F)c2)on1]

or a structurally similar compound.

In some embodiments, the pharmaceutical agent capable of inhibiting TDP-43 activity is

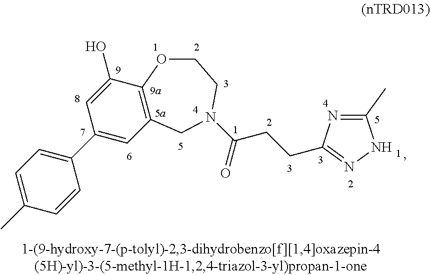

or a structurally similar compound.

In some embodiments, the pharmaceutical agent capable of inhibiting TDP-43 activity is

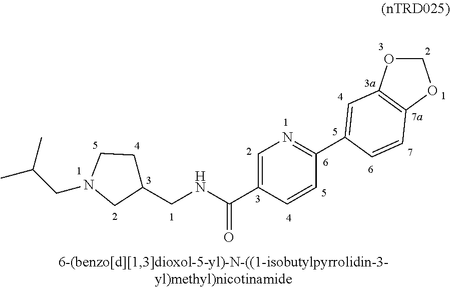 [SMILES: CC(C)CN1CCC(CNC(=O)c2ccc(-c3ccc4c(c3)OCO4)nc2)C1]

or a structurally similar compound.

In some embodiments, the pharmaceutical agent capable of inhibiting TDP-43 activity is

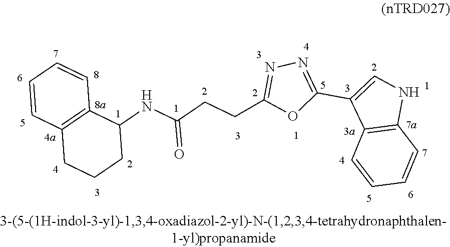 [SMILES: O=C(CCc1nnc(-c2c[nH]c3ccccc23)o1)NC1CCCc2ccccc21]

or a structurally similar compound.

In certain embodiments, the present invention provides methods for inhibiting TDP-43 activity in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for causing an allosteric modulation of the RNA binding domain (RRM) of TDP-43 in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for decreasing the ability of TDP-43 to bind RNA in a cell, comprising exposing the cell a composition comprising a pharmaceutical agent capable of inhibiting TDP-43 activity (e.g., nTRD22, nTRD013, nTRD025, nTRD027). In some embodiments, the cell is in culture. In some embodiments, the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

In certain embodiments, the present invention provides methods for treating, ameliorating and/or preventing a condition characterized with TDP-43 activity in a subject, comprising administering to the subject a composition comprising a pharmaceutical agent capable of inhibiting TDP-43 activity (e.g., nTRD22, nTRD013, nTRD025, nTRD027). In some embodiments, the subject is a human subject. In some embodiments, the subject is suffering from or at risk of suffering from a neurodevelopmental disorder. In some embodiments, the subject is a human subject suffering from or at risk of suffering from one or more of amyotrophic lateral sclerosis (ALS) or Alzheimer's disease (AD). In some embodiments, the method further comprises administration of a pharmaceutical agent known to be effective in treating a neurodevelopmental disorder (e.g., ALS, AD).

In certain embodiments, the present invention provides methods for treating, ameliorating and/or preventing one or more symptoms related to a condition characterized with TDP-43 activity in a subject, comprising administering to the subject a composition comprising a pharmaceutical agent capable of inhibiting TDP-43 activity (e.g., nTRD22, nTRD013, nTRD025, nTRD027). In some embodiments, the subject is a human subject. In some embodiments, the subject is suffering from or at risk of suffering from a neurodevelopmental disorder. In some embodiments, the subject is a human subject suffering from or at risk of suffering from one or more of amyotrophic lateral sclerosis (ALS) or Alzheimer's disease (AD). In some embodiments, the one or more symptoms include motor impairment. In some embodiments, the method further comprises administration of a pharmaceutical agent known to be effective in treating a neurodevelopmental disorder (e.g., ALS, AD).

In certain embodiments, the present invention provides kits comprising (1) a composition comprising a pharmaceutical agent capable of inhibiting TDP-43 activity (e.g., nTRD22, nTRD013, nTRD025, nTRD027), (2) a container, pack, or dispenser, and (3) instructions for administration.

Compositions within the scope of this invention include all compositions wherein the pharmaceutical agents which function as inhibitors of TDP-43 activity are contained in an amount that is effective to achieve its intended purpose. While individual needs vary, determination of optimal ranges of effective amounts of each component is within the skill of the art. Typically, the pharmaceutical agents which function as inhibitors of TDP-43 (e.g., small molecules, antibodies, mimetic peptides) may be administered to mammals, e.g. humans, orally at a dose of 0.0025 to 50 mg/kg, or an equivalent amount of the pharmaceutically acceptable salt thereof, per day of the body weight of the mammal being treated for disorders responsive to inhibition of TDP-43 activity. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the SGLT activity inhibiting agent. The unit dose may be administered one or more times daily as one or more tablets or capsules each containing from about 0.1 to about 10 mg, conveniently about 0.25 to 50 mg of the agent (e.g., mimetic peptide, small molecule) or its solvates.

In a topical formulation, the TDP-43 activity inhibiting agent (e.g., mimetic peptide, small molecule) may be present at a concentration of about 0.01 to 100 mg per gram of carrier. In a one embodiment, the inhibiting agent (e.g., mimetic peptide, small molecule) is present at a concentration of about 0.07-1.0 mg/ml, for example, about 0.1-0.5 mg/ml, and in one embodiment, about 0.4 mg/ml.

In addition to administering the TDP-43 activity inhibiting agent (e.g., mimetic peptide, small molecule) as a raw chemical, such inhibiting agents (e.g., mimetic peptides, small molecule) of the invention may be administered as part of a pharmaceutical preparation containing suitable pharmaceutically acceptable carriers comprising excipients and auxiliaries which facilitate processing of the TDP-43 activity inhibiting agents into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of a TDP-43 activity inhibiting agent (e.g., mimetic peptides, small molecules) of the invention. Foremost among such patients are mammals, e.g., humans, although the invention is not intended to be so limited. Other patients include veterinary animals (cows, sheep, pigs, horses, dogs, cats and the like).

The TDP-43 activity inhibiting agents (e.g., mimetic peptides, small molecules) and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C12). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EXPERIMENTAL

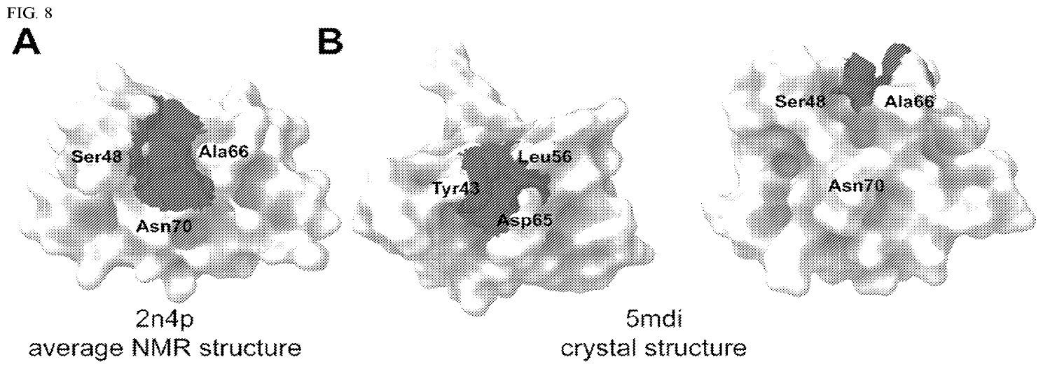

Experiments conducted during the course of developing embodiments for the present invention sough to target the NTD with small molecules using in-silico docking. Visual inspection of the surface showed no clear binding surface on both the X-ray (5mdi⁸) and NMR structures (2n4p¹²). The program SiteMap^{15,16} was used to determine if residues directly implicated in dimerization or residues nearby that could form a druggable pocket for binding of small molecules. A druggable pocket was identified by Sitemap on the NMR structure where residues S48, A66 and N70 are implicated (FIG. 8A). A slightly different site was found on the X-ray structure and surrounded the following residues: Y43, L56, and D65 (FIG. 8B).

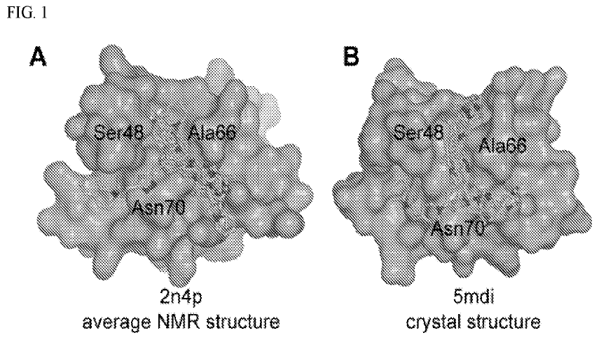

In subsequent experiments, a 50,000-compound library was then docked onto a grid of 6 angstroms surrounding the Sitemap pockets using Glide's virtual screening workflow. Even though the initial pockets were different on the NMR and crystal structure, compounds docked around S48, A66 and N70 (FIG. 1). A total of 20 compounds was chosen based on score and visual inspection: nTRD09-18 for the X-ray structure and nTRD19-nTRD28 for the NMR structure (Supplementary Table 1).

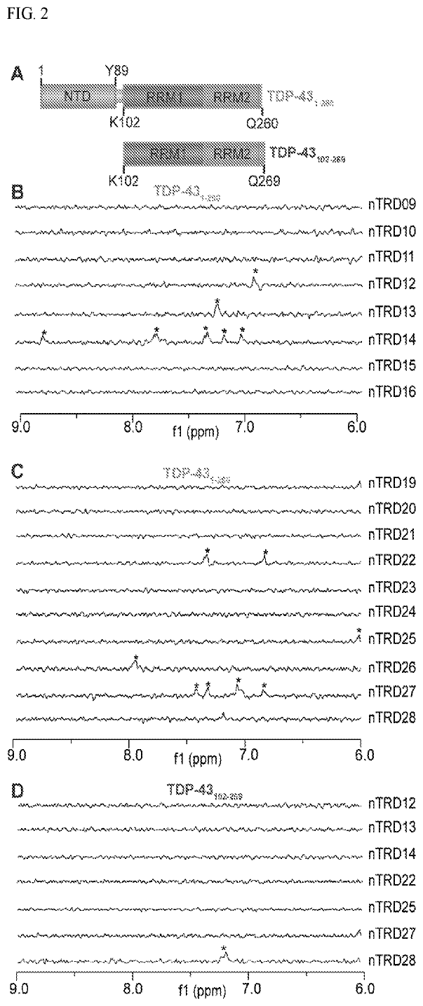

Saturation transfer difference nuclear magnetic resonance (STD-NMR) spectroscopy was then used to screen the binding of the compounds to TDP-43₁₋₂₆₀. As a negative control, binding to TDP43₁₀₂₋₂₆₉ lacking the NTD was also determined (FIG. 2A). nTRD12-14 and nTRD022

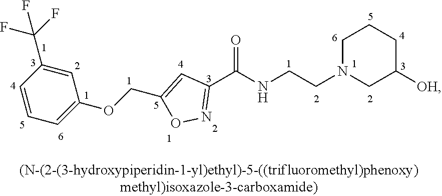 [SMILES: O=C(NCCN1CCCC(O)C1)c1cc(COc2cccc(C(F)(F)F)c2)on1]

nTRD025-TDP028 were observed as positive hits on TDP-43₁₋₂₆₀ that did not to TDP43₁₀₂₋₂₆₉ (FIG. 2B-D).

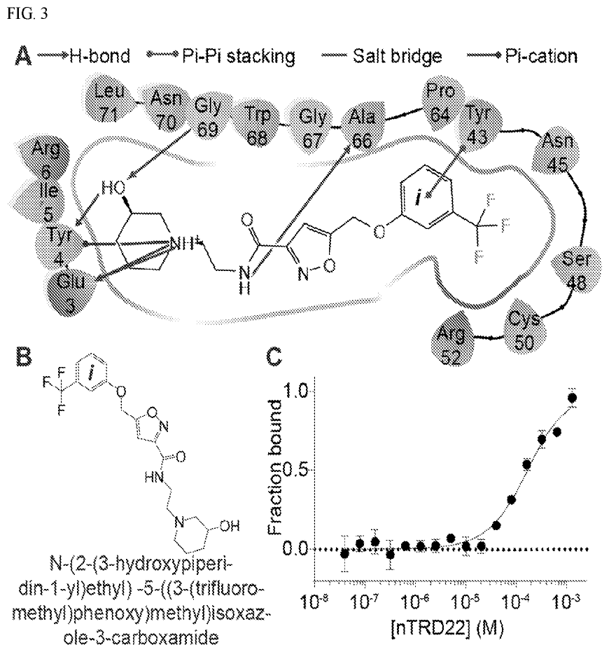

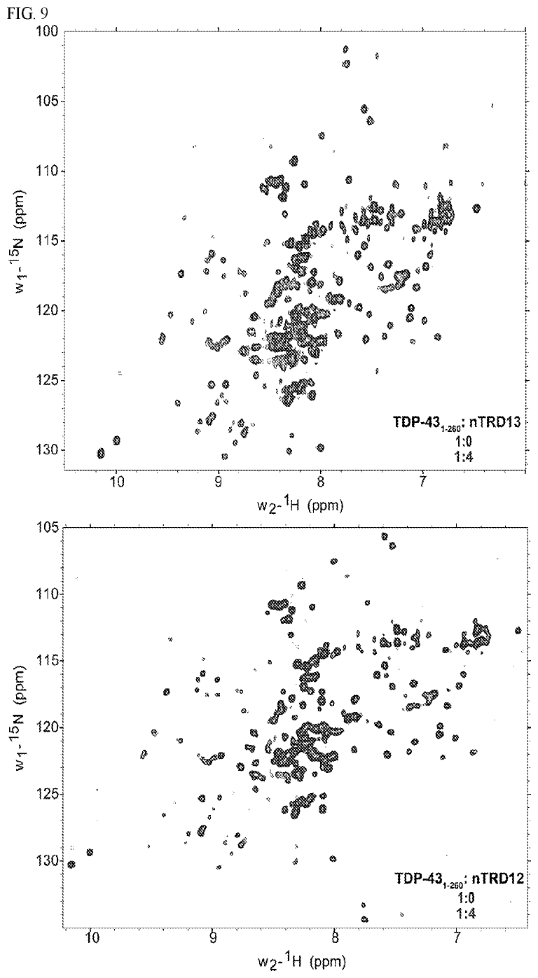

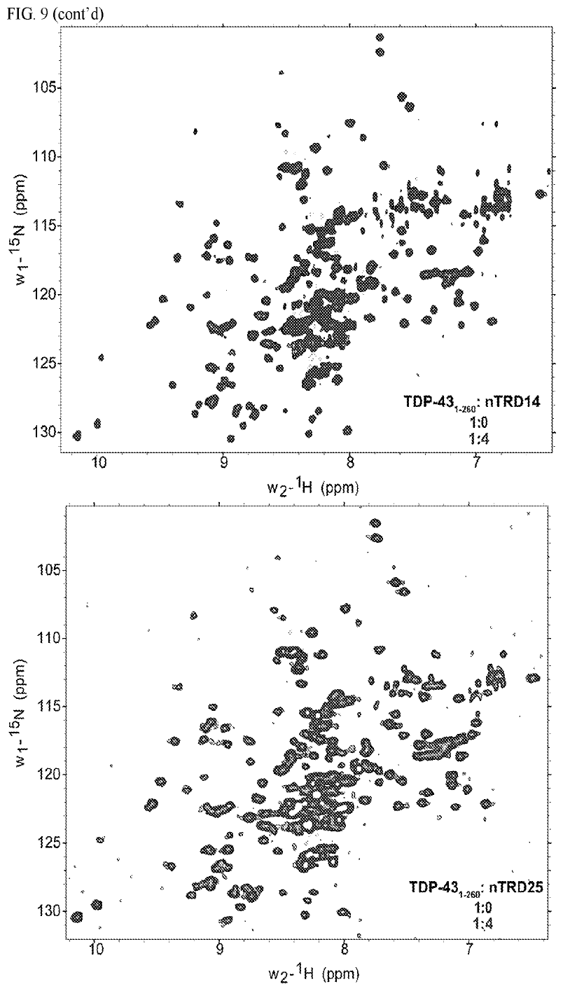

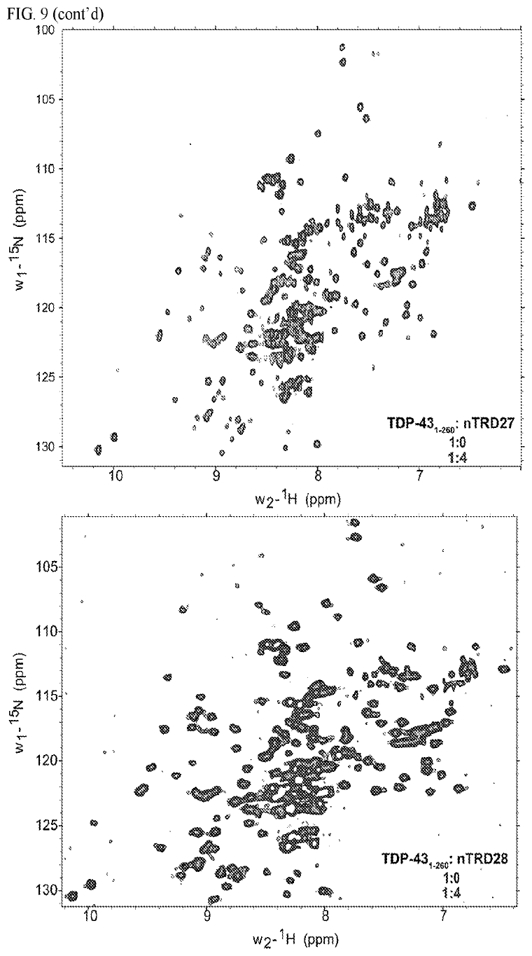

2D ¹⁵N-HSQC NMR spectrum of each positive hit was tested to further define the binding sites of the small molecule interactions with TDP-43 (FIG. 9). Using TDP-43₁₋₂₆₀, small molecules were added at a 4:1 molar ratio. Addition of 5 out of 6 of compounds either caused protein aggregation or showed little to no binding, possibly due to weak binding and were not further characterized. However, addition of nTRD022 (FIG. 3A,B) caused no protein aggregation with visible chemical shift perturbations and was hence chosen for further study.

In-silico docking of nTRD22 predicted interactions with Q3, Y43, S48, G59, and A66 (FIG. 3A). ¹H-NMR of nTRD22 was used to evaluate the on-resonance energy transfer from STD-NMR of nTRD22 with TDP-43₁₋₂₆₀. The peaks seen in the STD-NMR spectrum were in the region of the benzyl group (ii, 6.9-7.2 ppm) (FIG. 2C) confirming the I-stacking interaction of the benzene group of nTRD22 with Tyr43, predicted by virtual screening. Microscale thermophoresis (MST) measured an apparent Kd of 145±3 μM (FIG. 3C) for TDP-431-260.

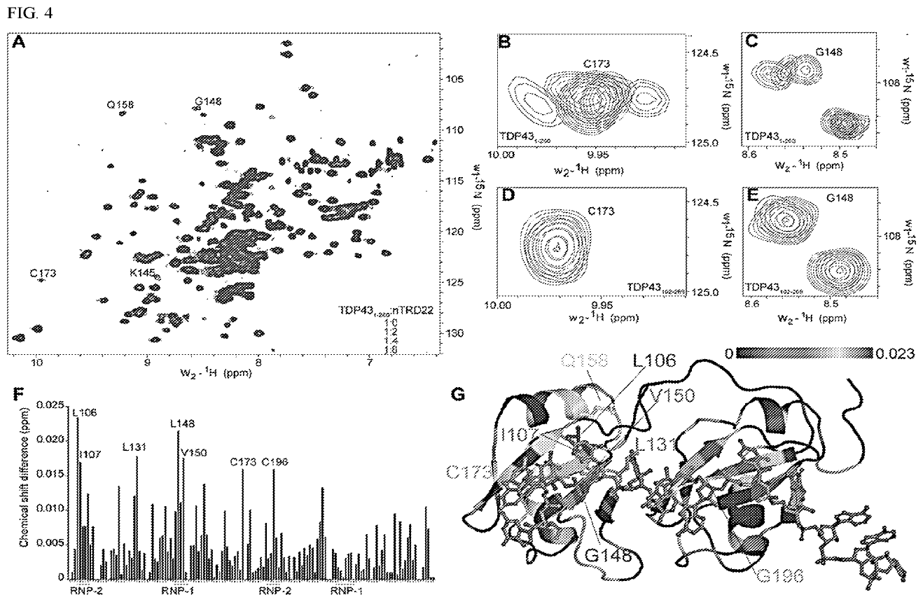

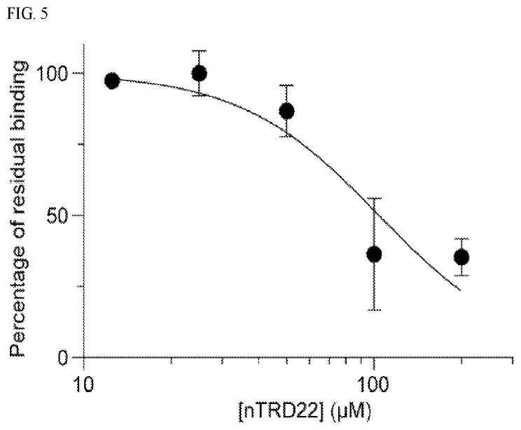

To further characterize where nTRD22 binds on TDP-43, perturbations were measured using a 2D ¹⁵N-HSQC NMR with increasing concentrations of nTRD22, up to a 8:1 molar ratio of nTRD22:TDP-43₁₋₂₆₀ (FIG. 4). Chemical shifts in the 2D ¹⁵N-HSQC of TDP-43₁₋₂₆₀ were observed, the majority of them occurring in the RRM region of the protein (FIG. 4). To determine if these shifts were due to direct binding of nTRD22 to TDP-43₁₀₂₋₂₆₉ (RRM region), a 2D ¹⁵HSQC-NMR on TDP43₁₀₂₋₂₆₉ with nTRD22 at a 4:1 molar ratio was collected (FIG. 4D, E). No shifts were observed in the spectra including the residues seen for the TDP-43₁₋₂₆₀ titration. nTRD22 was synthesized in house for further experiments (see supplementary methods) and was submitted to ¹H NMR and mass spectrometry for validation (see supplementary figures).

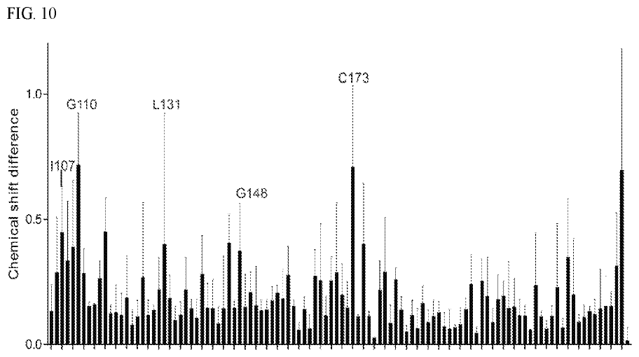

To define which residues in the RRM domains were affected, the chemical-shift perturbations (CSP) induced by nTRD22 were mapped onto the known structure of TDP-43₁₀₂₋₂₆₉ (PDB: 4bs2¹⁵) using a color gradient (FIG. 4F,G). Interestingly, in addition to peak broadening, several shifted residues are found to be implicated in RNA binding. More precisely, L106/1107 and G148/V150 are part of RNP-2 and RNP-1 of RRM1 respectively, while C196 is part of RNP-2 in RRM2¹⁶. Moreover, C173 is also shown to be affected in presence of RNA¹⁵. Replicates of this experiment showed similar profile of residues affected by nTRD22 (FIG. 10). It should be noted that in repeated experiments, different residues are shifted and although one representative spectrum is shown here (FIG. 4), the average shift from three experiments are also reported (FIG. 10). Although the shifts are small, RNP-2 remains consistently shifted even in the average CSP (FIG. 10); and since RNP sequences are highly conserved sequence motifs on TDP-43 required for nucleic acid recognition, CSP data suggests that nTRD22 might indirectly modulate TDP-43 RNA binding. To test this, an amplified luminescent proximity homogeneous (alpha) assay recently developed for another compound targeting TDP-43¹⁷ was used. TDP-43₁₋₂₆₀ binding to its canonical RNA sequence (UG6) was measured with ranging concentrations of compound and nTRD22 was able to inhibit 50% of the interaction with an IC₅₀ of ˜100 μM (FIG. 5).

Previous studies indicated that disruption of TDP-43 RNA binding, by expression of RNA binding deficient TDP-43, elicits the formation of intranuclear droplet-like structures and higher TDP-43 protein turnover¹⁸. Most importantly, the authors showed neuroprotective effects of RNA binding deficient TDP-43. Because nTRD22 is able to reduce TDP-43 binding to RNA, we hypothesized that nTRD22 could recapitulate some of the effects described with RNA binding deficient TDP-43.

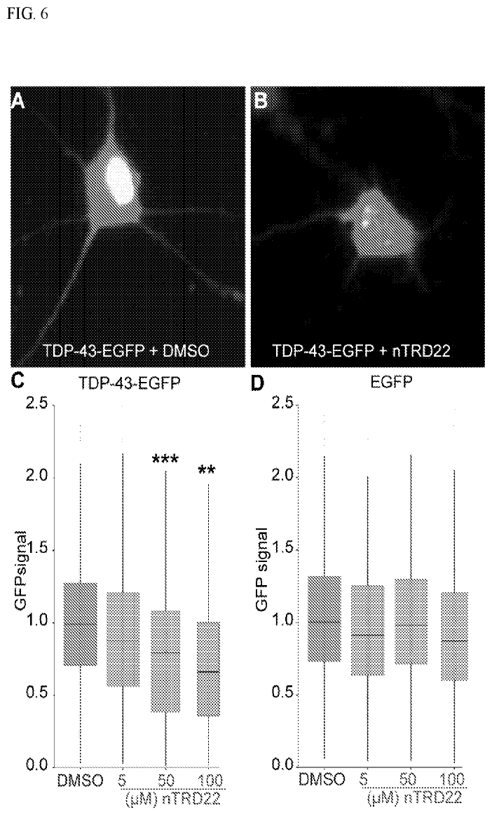

To test this, TDP-43 fused to enhanced green fluorescent protein (TDP43-EGFP) was expressed in rodent primary cortical neurons, applied nTRD22 at concentrations ranging from 5 to 100 μM, and imaged GFP positive cells by automated fluorescence microscopy¹⁸¹⁹. As expected, control cells incubated with DMSO exhibited a diffuse GFP signal, mainly in the nucleus (FIG. 6A). The formation of intranuclear TDP43-EGFP droplet-like structures in nTRD22 treated neurons was observed, similar to those formed by an RNA binding-deficient variant TDP43-EGFP¹⁸ (FIG. 6B). This was accompanied by a dose-dependent reduction in TDP43-EGFP steady state levels in treated neurons (FIG. 6C). No changes in the fluorescence of control cells transfected with EGFP was observed (FIG. 6D). Consistent with experiments in FIG. 5 demonstrating the effects of nTRD22 on the RNA binding property of TDP-43, these data suggest that nTRD22 likely elicits TDP43 phase separation and TDP-43 degradation by blocking RNA binding.

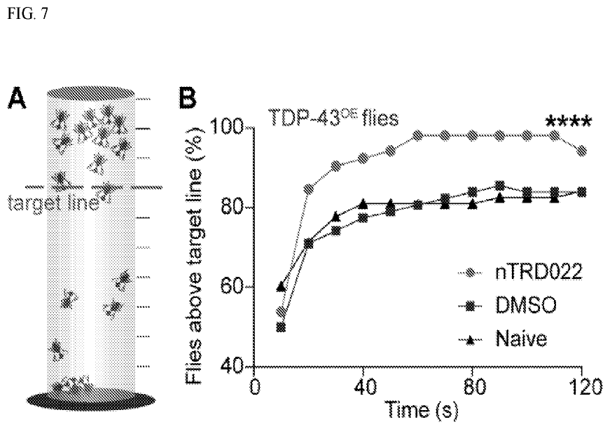

nTRD22 was also tested in a Drosophila overexpressing TDP-43 line, a well-known model of ALS¹⁷. The climbing assay was used, a behavior that measures motor strength and coordination in adult individuals and useful in many neurodegenerative disorders. Briefly, when flies are placed on a vial, their innate behavior is to attempt to climb to the top of the vial, a behavior called negative geotaxis. A sensitized version of the negative geotaxis assay that allows for earlier detection of milder defects over time was used according to²⁰ (FIG. 7A).

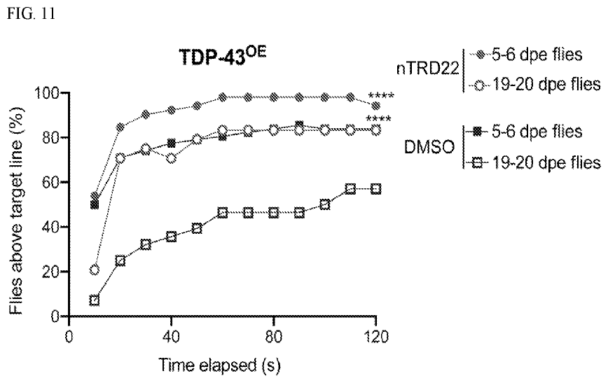

Flies expressing TDP-43 in motor neurons using the GAL4-UAS bipartite expression system exhibited locomotor defects in negative geotaxis (FIGS. 7 and 11). nTRD22 was significantly able to rescue climbing defects compared to DMSO treated or naive flies (FIG. 7B). This rescue effect was increased in aged flies (FIG. 11).

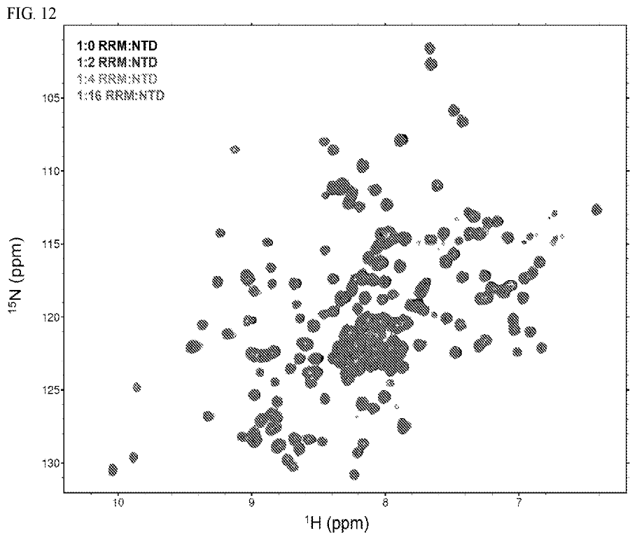

Experiments conducted during the course of developing embodiments for the present invention demonstrate that the ability of nTRD22 to modulate TDP-43 RNA binding in vitro has neuroprotective properties in a Drosophila model of ALS. However, the exact mechanism by which the compound modulates TDP-43 binding to RNA is still unclear. It was hypothesized that nTRD22 could influence residues important in RNA binding and allosterically modulate the RRM domain by (i) disrupting the binding of NTD to the RRM1 domain, leading to a modification of an open/closed conformation suggested by²¹ or (ii) modifying the orientation of RRM1 and RRM2 towards each other as previously discussed¹⁸. The first hypothesis was tested by measuring chemical shift perturbations using 2D ¹⁵N-HSQC NMR. The NTD portion of TDP-43, residues 1-102, was added to TDP-43₁₀₂₋₂₆₉ in a 8:1 molar ratio NTD:TDP-43₁₀₂₋₂₆₉. Data showed few significant perturbations related to NTD interacting with RRM domain (FIG. 12). Hence, it does not appear that there is significant interaction between TDP-43 NTD and the RRM. However, the other hypothesis stays open to further investigation.

The compound nTRD22 offers new opportunities as a tool to further study allostery within TDP-43 and validate reduction of RNA binding by chemical modulation as a possible neuroprotective avenue. Furthermore, nTRD22, by its unique property of allosteric modulation, makes it possible to target TDP-43 with possibly less off-targeting of RRM domains present in other RNA-binding proteins.

Materials. All reagents were purchased from Sigma (St. Louis, Mo., USA) and Fisher Scientific (Hampton, N.H.). nTRD22 (log P 2.4) has not been previously reported as an aggregator as predicted by Aggregator Advisor (see, Irwin, J. J.; et al., J Med. Chem. 2015, 58, 7076-7087). A similar query of nTRD22 in the Zinc15 database revealed no hits to molecules containing PAINS chemotypes. Human TDP-43₁₋₂₆₀ and TDP43₁₀₂₋₂₆₉ were expressed as described in our previous studies^{2,3}.

Molecular Modeling. Molecular docking studies were performed using Schrodinger suite of programs, Glide virtual screening workflow. X-ray structure of the N-terminal domain of TAR DNA-binding protein 43 (TDP-43) (PDB ID: 5mdi (see, Afroz, T.; et al., Nat. Commun. 2017, 8) and an average NMR structure (PDB ID: 2n4p (see, Mompein, M.; et al., FEBS J. 2016, 283, 1242-1260) were used for virtual screening of DIVERSet-CL, small molecule library of 50,000 compounds from ChemBridge. The top-ranked potential receptor binding sites were generated using siteMap screening for residues in N-terminal domain. Resulting docking poses were analyzed using XP G-score and the top compounds from X-ray structure, and from the NMR structure were selected for further screening.

Purification of recombinant TDP-43 N-terminal domain (NTD). TDP-43₁₋₁₀₂ was transformed into E. coli BL21 and then grown in LB media containing 50 μg/mL kanamycin at 37° C. overnight. Culture was inoculated into M9 media supplemented with uniformly labeled ¹⁵NH₄Cl (Cambridge Isotope Laboratories, Andover, Mass., USA). After the OD₆₀₀ reached 0.8, 1 mM isopropyl β-D-1-galactopyranoside was used to induce protein expression at 30° C. overnight. Cells were collected by 4500 rpm centrifugation and resuspended in 40 mM Tris-HCl, pH 7.5, 500 mM NaCl, 5 mM DTT, 30 mM imidazole and EDTA-free protease inhibitor cocktail. Cell disruption was performed by two rounds of high-pressure homogenization at 12,000 PSI with a LM10 Microfluidizer (Microfluidics, Westwood) and cell debris was removed by centrifugation at 34,000 rpm for 1 h at 4° C. Supernatant was then loaded on a His-Trap (GE Healthcare, Uppsala, Sweden) previously equilibrated using resuspension buffer. Protein was then eluted with a linear gradient of imidazole up to 400 mM. Eluted fractions of pure protein were dialyzed into NMR buffer (40 mM HEPES, pH 6.5, 500 mM NaCl, 4 mM DTT). Dialyzed protein was concentrated with Amicon Ultra 15 centrifugal filters (Regenerated cellulose 3,000 NMWL; Merck Millipore, Darmstadt, Germany).

Saturation Transfer Difference Nuclear Magnetic Resonance (STD-NMR). Spectroscopy for small molecule binding. 1D ¹H STD-NMR was performed exactly as previously described (see, Frangois-Moutal, L.; et al., ACS Chem. Biol. 2019, 14, 2006-2013; Frangois-Moutal, L.; et al., ACS Chem. Biol. 2018, 13, 3000-3010).

Heteronuclear Single Quantum Correlation-NMR (HSQC-NMR). NMR spectra were acquired in 40 mM HEPES pH 6.5, 500 mM NaCl, 4 mM DTT with 10% D20 at 100 μM protein concentrations using 5 mm Shigemi NMR tubes. NMR data were collected at 298 K on a Bruker Avance NEO 600 MHz and 800 MHz spectrometers with TCI-H&F/C/N probe. NMR processing and analysis was done using NMRpipe, NMRDraw and NMRFAM-Sparky. Transverse relaxation optimized spectroscopy (TROSY) was used for all HSQC experiments.

Heteronuclear Single Quantum Correlation-NMR (HSQC-NMR) of NTD-RRM TDP-43. NTD as well as ¹⁵N-labeled RRM aliquots were dialyzed against the following buffer: 40 mM HEPES, pH 6.5, 500 mM NaCl, for 3 hours in cold room before exchanging with fresh buffer and continuing dialysis overnight. All spectra were collected at 298 K on a 500 MHz Bruker Avance spectrometer equipped with TCI cryoprobe running TopSpin 1.3 software. Samples were individually shimmed and tuned, and carrier position and 90-degree pulse length were optimized for each sample. Each titration point consisted of ¹H 1-D WaterGATE (3-9-19) spectrum [td=2048, ns=32] as well as a ¹⁵N-¹H 2-D BEST-TROSY [td=1024*128, o3p=116 ppm, sw=l4 ppm, 32 ppm, dl=180 ms]. The number of scans was adjusted to account for changes in ¹⁵N-labeled RRM concentration (64, 256, 1024, 1792 for the four titration points, respectively). Data were processed in NMRPipe (PMID: 8520220) and visualized in NMRFAM-SPARKY (PMID: 25505092).

Microscale Thermophoresis (MST). Microscale thermophoresis experiments were performed using a NanoTemper Monolith Instrument (NT.115), similarly to Frangois-Moutal, L.; et al., ACS Chem. Biol. 2019, 14, 2006-2013.

Amplified luminescent proximity homogeneous alpha assay (ALPHA). Amplified luminescent proximity homogeneous alpha assay (ALPHA) experiment was conducted as reported before (see, Frangois-Moutal, L.; et al., ACS Chem. Biol. 2019, 14, 2006-2013). TDP-43₁₋₂₆₀-His (0.75 nM) and a single concentration of biotinylated-UG6 RNA (0.6 nM) with increasing concentration of nTRD22 were used.

Cell culture and transfection. Rodent primary cortical neurons were prepared as before (see, Flores, B. N.; et al., Cell Rep. 2019, 27, 1133-1150.e8; Malik, A. M.; et al., Elife 2018, 7; Weskamp, K.; et al., J. Vis. Exp. 2019). Briefly, neurons dissected from embryonic day 20-21 Long Evans rat pups were cultured at a density of 0.6×10⁶ cells ml⁻¹ in 96 well plates coated with laminin (Corning) and D-polylysine (Millipore). Primary neurons were transfected with plasmids 4 days after plating using Lipofectamine 2000 (Invitrogen) as described in Malik, A. M.; et al., Elife 2018, 7; Weskamp, K.; et al., J. Vis. Exp. 2019. rTRD022 or vehicle was added 24 h after transfection, immediately following the first imaging run.

Fluorescence microscopy. Primary cortical neurons were imaged using an automated microscopy platform described previously (see, Flores, B. N.; et al., Cell Rep. 2019, 27, 1133-1150.e8; Malik, A. M.; et al., Elife 2018, 7; Weskamp, K.; et al., J. Vis. Exp. 2019). Briefly, images were obtained with an inverted Nikon TiE-2000 microscope equipped with a Nikon PerfectFocus 3 system, a high-numerical aperture 20× objective lens, Chroma ET Sedat filter sets, Sutter 4-2-1 multiLED system, and Andor Zyla 4.2+ sCMOS camera. Stage movements were accomplished using an ASI 2000 stage with rotary encoders in the x- and y-axes. The microscope and associated components were encased in a climate-controlled environmental chamber built specifically for this purpose. The illumination, filter wheels, focusing, stage movements, and image acquisitions were fully automated and coordinated with publicly available (ImageJ, pManager) software.

Image analysis. Custom ImageJ/Fiji macros and Python scripts (described in PMC6050042) were used to identify neurons and draw regions of interest (ROIs) based upon size, morphology, and fluorescence intensity. Additional scripts were used to identify and draw regions of interest (ROIs) surrounding each cell, and measure fluorescence intensity within individual ROIs. Boxplots were generated using the ggplot package in R, and statistical comparisons accomplished using one-way ANOVA with Tukey's test.

Drosophila genetics. Flies were raised at 23° C. on standard cornmeal medium with a 12/12-hrs light dark cycle, 60-80% relative humidity. The following fly stocks obtained from Bloomington Drosophila Stock Center (Bloomington, Ind.) were used in this study: w[1118]; P{w[+mC]=UAS-TDP-43.YFP}8S (BDSC #79589, called TDP-43^{OE}), w[*]; P{w[+mW.hs]=GawB}D42 (BDSC #8816, called D42 Gal4), and w[1118] (BDSC #5905, called w118). Transgenic expressing human TDP-43 fly (PMID: 20740007) was crossed to D42 Gal4 fly to drive its expression in motor neurons. For genetic control, w1118 (the parental strain used in the generation of TDP-43 transgenic line) was crossed to TDP-43^{OE}. A mixture of both male and female adults was used throughout.

Climbing assay. Fly crosses were made in an egg-laying cage with a removable egg-laying dish. The egg laying dish consisted of a 60×15 mm petri dish containing sucrose agar medium spotted with a fresh dab of yeast paste. Flies were allowed to lay eggs for 24 hours at room temperature. Eggs were collected using a fine paintbrush dampened with water and gentle brushing of the plate to dislodge eggs from the agar. Eggs were then transferred to cornmeal food vials containing either Dimethyl sulfoxide (DMSO 0.05%) solvent or nTRD022 (50 μM). After eclosion, the adult flies were transferred to a fresh supplemented cornmeal food vial. Climbing assay experiments were performed as described in PMID: 26132637. On the day of the experiment, flies were transferred from a single vial into a 250-mL glass graduated cylinder that was sealed with a wax film to prevent escape. Flies were tapped down to the bottom of the cylinder and their climbing behavior was captured using a video camera (Samsung HMX-F90 HD) for 2 min. The number of flies crossing the height of 17.5 cm (190 mL graduation) was manually scored. To avoid variation due to circadian rhythms experiments (for both genotypes) were conducted at the same time of day and in ambient light.

### Synthesis of 5-((3-(trifluoromethyl)phenoxy)methyl)isoxazole-3-carboxylic acid

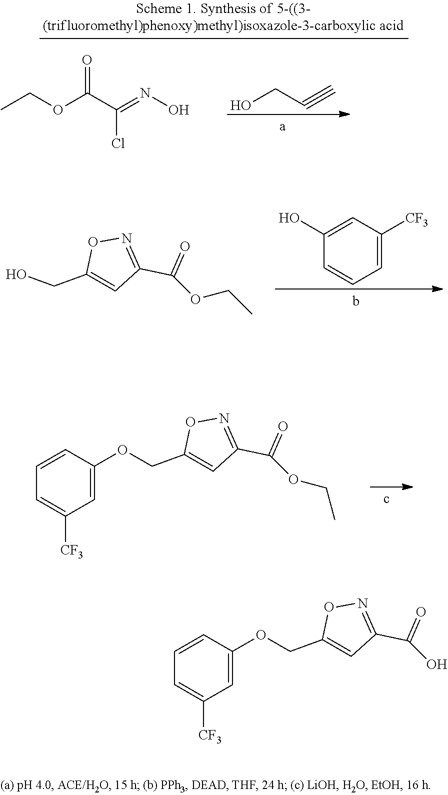

Intermediate ethyl 5-(hydroxymethyl)isoxazole-3-carboxylate: Ethyl (Z)-2-chloro-2-(hydroxyimino)acetate (12 mmol) and prop-2-yn-1-ol (10 mmol) were dissolved in acetone (5 mL, in a round bottom flask). The mixture was stirred at room temperature and was stepwise added 100 mL of 0.1 M phosphate buffer for 15 h. Then the mixture was extracted with EtOAc (15 mL) for 3 times. Organic extracts were combined, dried with Na₂SO₄, and evaporated under reduced pressure to obtain a crude product. The product was purified by silica gel column chromatography using hexane/ethyl acetate as eluents to give light yellow oil (1.01 g, yield: 62%).

Intermediate ethyl 5-((3-(trifluoromethyl)phenoxy)methyl)isoxazole-3-carboxylate: The solution obtained by adding ethyl 5-(hydroxymethyl)isoxazole-3-carboxylate (6.4 mmol) and 3-(trifluoromethyl)phenol (12.8 mmol) to dehydrated THF at 0° C. Triphenylphosphine (12.8 mmol), triethylamine (12.8 mmol) and DIED (9.6 mmol) were added to solution under nitrogen protection. The reaction mixture was warmed to room temperature and stirred for 2 hours, then poured into water, and the mixture was extracted twice with ethyl acetate. The organic layer was washed with saturated aqueous brine, dried over Na₂SO₄ and then concentrated under pressure. The product was purified by silica gel column chromatography using hexane/ethyl acetate as eluents to give light white solid (1.56 g, yield: 77%).

Intermediate 5-((3-(trifluoromethyl)phenoxy)methyl)isoxazole-3-carboxylic acid: Ethyl 5-((3-(trifluoromethyl)phenoxy)methyl)isoxazole-3-carboxylate (5 mmol) was added to ethanol (15 mL), and LiOH (15 mmol) and H₂O (6.5 mL) were further added thereto. The mixture was stirred at room temperature for 16 hours and then concentrated under reduced pressure. Dilute hydrochloric acid was added to the concentrate, the mixture was cooled to 0° C., and the precipitated solid was filtered. The resulting solid was dried under reduced pressure to give 1.11 g of product (yield: 75%).

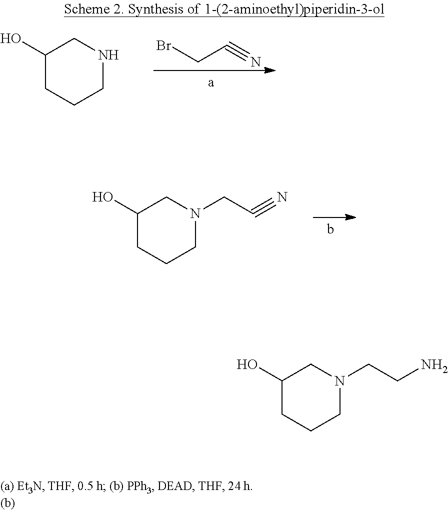

Intermediate 2-(3-hydroxypiperidin-1-yl)acetonitrile: Bromoacetonitrile (10 mmol) was added dropwise to a solution of 3-hydroxypiperidine (10 mmol) in dry THF (25 mL) under N2, while the temperature was maintained between 45 and 50° C. Following addition of bromoacetonitrile, the solution was refluxed for 30 min, before allowing the solution to cool to room temperature. The solvent was removed in vacuo and the residual oil was purified by flash chromatography using CH₂Cl₂/CH₃OH as eluent. The title compound was obtained as a straw-colored oil (80 mg, yield: 57%).

Intermediate 1-(2-aminoethyl)piperidin-3-ol: LiAlH₄ (20 mmol) was added to dry THF (20 mL) at 0° C. in a three-neck round bottom flask under N2. The solution was stirred for 15 min before the 3-hydroxypiperidin-1-ylacetonitrile (5 mmol), diluted in dry THF (5 mL), was added slowly via syringe. The reaction mixture was then refluxed for 5 h before allowing the solution to cool to room temperature. Excess LiAlH₄ was destroyed by dropwise addition of 2.4 mL of H₂O and 2.4 mL of NaOH (15%), and finally EtOAc was added dropwise until no effervesence was observed. The formed granular precipitate (lithium hydroxide and aluminum hydroxide) was filtered off and washed several times with CH₂Cl₂ and EtOAc. The organic layer was dried (MgSO₄) and the solvent was removed in vacuo to yield a thick yellowish oil. the residual oil was directly used in next step without purification (53 mg, yield: 64%).

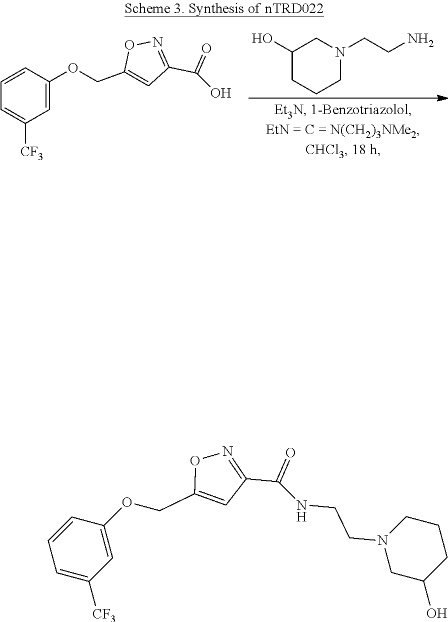 [SMILES: O=C(O)c1cc(COc2cccc(C(F)(F)F)c2)on1 ; NCCN1CCCC(O)C1 ; O=C(NCCN1CCCC(O)C1)c1cc(COc2cccc(C(F)(F)F)c2)on1]

Synthesis of nTRD022: 5-((3-(Trifluoromethyl)phenoxy)methyl)isoxazole-3-carboxylic acid (0.4 mmol), 1-(2-aminoethyl)piperidin-3-ol (0.5 mmol), triethylamine (0.5 mmol) and 1-hydroxybenzotriazole (0.05 mmol) were added to chloroform (2 mL). 1-Ethyl-3-(3-dimethylaminopropyl) carbodiimide hydrochloride (0.5 mmol) was added to the mixture at room temperature, and the mixture was stirred overnight and then concentrated under reduced pressure. Dilute hydrochloric acid was added to the concentrate, and the mixture was extracted twice with ethyl acetate. The organic layer was washed with saturated saline water, dried over anhydrous sodium sulfate, and then concentrated under reduced pressure. The residue was applied to a silica gel column chromatography to obtain nTRD022 (91 mg, yield: 57%). ¹H NMR (400 MHz, Chloroform-d) δ 7.46-7.36 (m, 1H), 7.26 (ddt, J=7.7, 1.6, 0.8 Hz, 1H), 7.17 (t, J=2.1 Hz, 1H), 7.13-7.07 (m, 1H), 6.78 (s, 1H), 5.20 (d, J=0.8 Hz, 2H), 3.81 (dp, J=6.3, 3.2 Hz, 1H), 3.51 (p, J=5.7 Hz, 2H), 2.56 (t, J=6.1 Hz, 4H), 2.40 (d, J=27.8 Hz, 5H), 1.85-1.74 (m, 2H), 1.71-1.61 (m, 1H), 1.58-1.47 (m, 2H). HRMS (EI) m/z [M+H]+ calculated for C19H22F3N304: 414.16352, found 414.16351.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A composition comprising a pharmaceutical agent capable of inhibiting TDP-43 activity.

2. The composition of claim 1, wherein the pharmaceutical agent capable of inhibiting inhibiting TDP-43 activity is capable of one or more of the following: binding the N-terminal domain of TDP-43, engaging a pocket within the TDP-43 characterized by amino acids S48, A66, and N70, causing an allosteric modulation of the RNA binding domain (RRM) of TDP-43 thereby decreasing the ability of TDP-43 to bind RNA, and mitigating motor impairment in a subject suffering from or at risk of suffering from motor impairment.

3. The composition of claim 1, wherein the pharmaceutical agent is a small molecule, antibody, or mimetic peptide.

4. The composition of claim 1, wherein the pharmaceutical agent is selected from 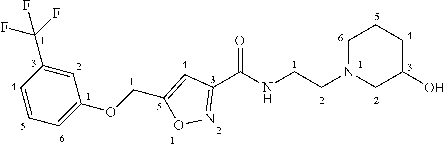 [SMILES: O=C(NCCN1CCCC(O)C1)c1cc(COc2cccc(C(F)(F)F)c2)on1]
N-(2-(3-hydroxypiperidin-1-yl)ethyl)-5-((3-(trifluoromethyl)phenoxy)methyl)isoxazole-3-carboxamide or a pharmaceutically acceptable salt thereof and/or a structurally similar compound, 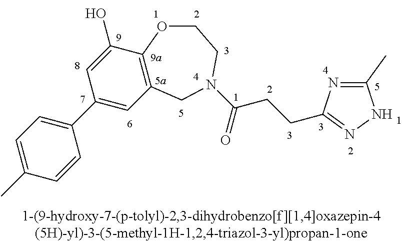
or a pharmaceutically acceptable salt thereof and/or a structurally similar compound, 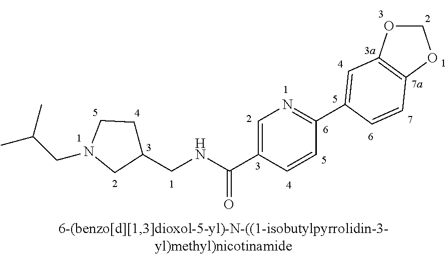 [SMILES: CC(C)CN1CCC(CNC(=O)c2ccc(-c3ccc4c(c3)OCO4)nc2)C1]
or a pharmaceutically acceptable salt thereof and/or a structurally similar compound, and 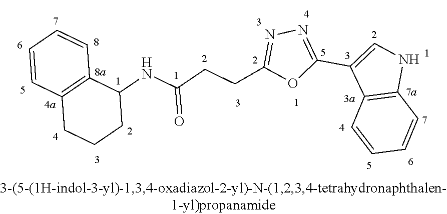 [SMILES: O=C(CCc1nnc(-c2c[nH]c3ccccc23)o1)NC1CCCc2ccccc21]
or a pharmaceutically acceptable salt thereof and/or a structurally similar compound.

5. A method for inhibiting TDP-43 activity in a cell, comprising exposing a composition of claim 1 to the cell.

6. The method of claim 5, wherein the cell is in culture.

7. The method of claim 5, wherein the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

8. A method for causing an allosteric modulation of the RNA binding domain (RRM) of TDP-43 in a cell, comprising exposing a composition of claim 1 to the cell.

9. The method of claim 8, wherein the cell is in culture.

10. The method of claim 8, wherein the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

11. A method for decreasing the ability of TDP-43 to bind RNA in a cell, comprising exposing a composition of claim 1 to the cell.

12. The method of claim 11, wherein the cell is in culture.

13. The method of claim 11, wherein the cell is a living cell in a subject (e.g., a human subject) (e.g., a human subject suffering from or at risk of suffering from a neurodevelopmental disorder) (e.g., a human subject suffering from or at risk of suffering from one or more of amyotrophic lateral sclerosis (ALS) or Alzheimer's disease (AD)) (e.g., a human subject suffering from or at risk of suffering from a condition characterized with TDP-43 activity).

14. A method for treating, ameliorating and/or preventing a condition characterized with TDP-43 activity in a subject, comprising administering to the subject a composition of claim 1.

15. The method of claim 14, wherein the subject is a human subject.

16. The method of claim 14, wherein subject is suffering from or at risk of suffering from a neurodevelopmental disorder (e.g., ALS, AD, Parkinson's disease).

17. The method of claim 14, wherein subject is suffering from or at risk of suffering from motor impairment.

18. A kit comprising (1) a composition comprising a composition of claim 1, (2) a container, pack, or dispenser, and (3) instructions for administration.

19. A pharmaceutical composition comprising a composition as recited in claim 1.
